(19) United States
(12) Patent Application Publication
(10) Pub. No.: US 2024/0016426 A1
(43) Pub. Date: Jan. 18, 2024

(54) System and Methods for At-Home Diagnostics Utilizing Microneedles

(71) Applicant: IDEXX Laboratories, Inc., Westbrook, ME (US)

(72) Inventors: Murthy VSN Yerramilli, Scarborough, ME (US); Justin Griffin, Scarborough, ME (US)

(21) Appl. No.: 18/352,792

(22) Filed: Jul. 14, 2023

Related U.S. Application Data
(60) Provisional application No. 63/389,562, filed on Jul. 15, 2022.

(51) Int. Cl. A61B 5/15; C12Q 1/32; G01N 33/72

(52) U.S. Cl. CPC A61B 5/150984; A61B 5/150114; A61B 5/150755; C12Q 1/32; G01N 33/72; A61B 2503/40; G01N 2333/902

(57) ABSTRACT

A method of analyzing a blood sample from an animal is disclosed. The method includes: (a) extracting a blood sample from a blood collection device, wherein the blood collection device comprises: (i) a microneedle array and (ii) a blood storage layer, and wherein the microneedle array transports the blood sample from the animal to the blood storage layer, and wherein the microneedle array comprises one or more microneedles; (b) conducting at least one test on the extracted blood sample; and (c) reporting results from the at least one test to a customer.

This application claims the benefit of U.S. Provisional Application No. 63/389,562 filed on Jul. 15, 2022, which is incorporated by reference herein its entirety.

### FIELD OF THE DISCLOSURE

The present disclosure involves systems and methods for analyzing a blood sample from an animal. Namely, devices and methods of the disclosure collect blood from an animal utilizing a microneedle array of one or more microneedles embedded in a blood collection device.

### BACKGROUND

Microneedles can be utilized for a variety of different tasks, including tasks relating to collecting a blood sample from an animal.

### SUMMARY

In particular, microneedles can be used to pierce the skin of an animal, pass the interstitial fluid, and transport blood from the animal to a collection device. Further, an array of microneedles can be utilized to simultaneously collect blood while reducing collection time without enhancing pain or discomfort experienced by the animal as compared to large, conventional needles.

When blood is drawn from an animal to perform one or more tests (e.g., blood tests), often the blood draw is conducted at a particular location, such as a veterinary office. More particularly, intravenous blood draws with a conventional needle may require specialized training to locate and access appropriate veins of the animal, and may be performed by veterinarians or veterinary technicians at a veterinary office. However, animals are not always comfortable, responsive, and/or cooperative at unfamiliar locations (e.g., a veterinary office) and/or with one or more parties administering the blood draw (e.g., a veterinary technician or veterinarian). Also, conventional needles may draw large amounts of fluids (e.g., blood) from the animal to perform the tests. Accordingly, existing testing devices and procedures for animals can be difficult to execute, as well uncomfortable and invasive to the animal.

The present disclosure is directed to systems and methods for testing blood from non-human animals utilizing microneedles. Because the systems and methods of the present disclosure utilize microneedles, minimal training may be required for a user to draw blood from the animal, and the blood draw can be conducted by users familiar to the animal, such as the animal's caretaker. Moreover, because the blood draw can be conducted by users with minimal training, the blood draw can be conducted in any suitable location, such as the animal's home or other location familiar to the animal.

The present disclosure is directed to, in example embodiments, methods, devices, and systems for analyzing a blood sample from an animal that comprise extracting a blood sample from a blood collection device, the blood collection device including a microneedle array and a blood storage layer. Because microneedles are utilized in the blood collection device, a much smaller blood sample may be collected. In some embodiments, the blood collection device includes an outer layer (which may restrict movement of the one or more microneedles in the microneedle array), a compressible button configured to provide haptic feedback to a user, a peel-to-expose packaging, a clamping device with a biasing member, a wearable sleeve, and/or a removable cover for the blood storage layer. In example embodiments, the blood storage layer may also seal around and/or store the blood sample until analysis can be performed on the blood sample. The results of these tests may be reported to the party responsible for the animal.

In an example, a method of analyzing a blood sample from an animal is disclosed. The method comprises extracting a blood sample from a blood collection device, wherein the blood collection device comprises: (i) a microneedle array; and (ii) a blood storage layer, and wherein the microneedle array transports the blood sample from the animal to the blood storage layer, and wherein the microneedle array comprises one or more microneedles. The method also comprises conducting at least one test on the extracted blood sample. The method also comprises reporting results from the at least one test to a customer.

In another example, a blood collection device for collecting a blood sample from an animal is disclosed. The blood collection device comprises: (i) a microneedle array, wherein the microneedle array comprises one or more microneedles; (ii) an outer layer surrounding one or more microneedles of the microneedle array, and wherein the outer layer restricts movement of the one or more microneedles of the microneedle array with respect to the blood storage layer; and (iii) a blood storage layer, wherein the microneedle array transports the blood sample from the animal to the blood storage layer.

In another example, a blood collection device for collecting a blood sample from an animal is disclosed. The blood collection device comprises: (i) a microneedle array, wherein the microneedle array comprises one or more microneedles; (ii) a blood storage layer, wherein the microneedle array transports the blood sample from the animal to the blood storage layer; and (iii) a wearable sleeve, wherein the sleeve injects the microneedle array into an appendage of the animal via an exterior surface of the wearable sleeve.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The above, as well as additional, features will be better understood through the following illustrative and non-limiting detailed description of example embodiments, with reference to the appended drawings.

FIG. 1A illustrates an exploded view of a microneedle array, according to an example embodiment.

FIG. 1B illustrates an assembled view of the microneedle array of FIG. 1A, according to an example embodiment.

FIG. 1C illustrates an enlarged section view of the microneedle array of FIG. 1A, according to an example embodiment.

FIG. 1D illustrates an enlarged section view of a microneedle array, according to an example embodiment.

FIG. 1E illustrates a microneedle in isolation, according to an example embodiment.

FIG. 1F illustrates an exploded view of a microneedle array, according to an example embodiment.

FIG. 1G illustrates an assembled view of the microneedle array of FIG. 1F, according to an example embodiment.

FIG. 1H illustrates an assembled view of a microneedle array, according to an example embodiment.

FIG. 1I illustrates an enlarged section view of the microneedle array of FIG. 1H, according to an example embodiment.

FIG. 2 illustrates a blood collection device including a microneedle array, according to an example embodiment.

FIG. 3A illustrates another blood collection device including a microneedle array, according to an example embodiment.

FIG. 3B illustrates a side view of the blood collection device of FIG. 3A, according to an example embodiment.

FIG. 4A illustrates bottom perspective view of another blood collection device, according to an example embodiment.

FIG. 4B illustrates a top perspective view of the blood collection device of FIG. 4A, according to an example embodiment.

FIG. 4C illustrates another top perspective view of the blood collection device of FIG. 4A with a removable layer removed, according to an example embodiment.

FIG. 4D illustrates the blood collection device of FIG. 4A on a user, according to an example embodiment.

FIG. 5 illustrates a flowchart of one method of collecting a blood sample from an animal, according to an example embodiment.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example.

When blood from an animal subject is drawn to be analyzed (e.g., for a blood test), often these draws are performed on the animal at a particular location and/or by a particular party (e.g., such as by bringing an animal to a veterinary office or bringing a travelling veterinarian to an animal). However, animals are not always comfortable, responsive, and/or cooperative in this process.

For instance, some animals can experience large amounts of stress when being taken out of their ordinary environments. Additionally, most veterinary offices are suited to treat multiple animals at the same time, and some animals do not interact well with other animals. Because many veterinary offices treat multiple animals at the same time, it is likely that an animal being brought in to a veterinary office will interact with another animal also present at the veterinary office. Further, some animals are difficult to transport due to their size, shape, weight, or other factors.

Alternatively, requiring a vet or technician to travel to an animal in its own environment for testing can be expensive and difficult to obtain. Such traveling vets and technicians are able to see less animals than if they are located in a fixed location (e.g., at a veterinary office) for a variety of reasons, including time needed to travel from one appointment to the next. Therefore, the demand and expense for utilizing these parties are increased and result in less animals being treated.

Further, some animals do not interact well with unfamiliar individuals, such as a veterinary doctor or a veterinary assistant. Often, an animal is more comfortable interacting with the person associated with and/or responsible for that animal.

Lastly, conventional intravenous needles draw a significant amount of blood from the animal. Such procedures may present a number of additional concerns. For example, if a large blood sample is withdrawn using a large needle, then the animal may experience pain, discomfort, and irritation. Accordingly, such blood tests can be difficult to execute and invasive to the animal.

To help address these issues, embodiments of the present disclosure include a microneedle array that can be utilized to collect a blood sample from an animal without being as offensive and destabilizing to the animal as the conventional blood draws previously described. An array of microneedles, which is less invasive than a conventional intravenous needle, may be used to carry blood into a blood storage layer of a blood collection device. The array of microneedles may be able to be used at home by a party or customer associated with the animal, and that the animal is more comfortable around. For instance, the blood sample may be collected by a user associated with and/or responsible for the animal, stored in the blood storage layer of the device, and then withdrawn from the device and analyzed at a later time.

In some embodiments, the microneedle array includes a plurality of microneedles. Without being bound by theory, each microneedle can draw a predetermined volume of blood over a predetermined time. Accordingly, the number of microneedles in the microneedle array can be selected to draw a predetermined amount of blood over a predetermined amount of time, the predetermined amount of blood being sufficient to perform one or more blood tests. These collection times could be in the range of 30 seconds to 5 minutes, depending on the number of microneedles, the type of animal, and/or the amount of blood to be withdrawn, among other factors.

In some examples, the blood storage layer is made of an absorbent material structurally configured to store dried blood, defines a chamber designed to store liquid blood, or a combination of the two. For instance, blood can be drawn by and through the microneedle array by one or more factors, including the animal's blood pressure pumping blood through the appendage in which the microneedle array is inserted, osmotically by the absorbent material, and/or other factors.

In some embodiments, the blood collection device includes a peel-to-expose package, a clamping device, and/or a wearable sleeve to allow a user associated with and/or responsible for an animal to more easily collect a blood sample from the animal using the array of microneedles, or any suitable combination thereof, among other possibilities. For instance, the peel-to-expose package may be used to selectively cover the microneedle array and the blood storage layer such that the microneedle array and/or the blood storage layer are exposable to use the device and resalable when the collection has been completed. In some embodiments, the blood collection device may include a clamping device that biases the microneedle array into the appendage of an animal and secures itself to the appendage via a biasing member. In some embodiments, the blood collection device includes a wearable sleeve with the microneedle array accessible from and positioned on an exterior surface of the wearable sleeve.

In another aspect of the disclosure, an example method may include measuring one or more activities of an enzyme in a blood sample. The term “activity of an enzyme,” as used herein, generally refers to a reaction that catalyzes the conversion of substances in the sample to produce reaction products such as redox products.

In some examples, the blood sample may include a lysed blood sample. In a further aspect, in some embodiments of the disclosure as described herein, glucose-6-phosphate dehydrogenase (G6DPH) may be the enzyme. As would be understood by the person of ordinary skill in the art, G6PDH catalyzes the oxidation of D-glucose-6-phosphate to 6-phosphogluconolactone and reduction of NADP⁺ to NADPH. NADP⁺ is nicotinamide adenine dinucleotide phosphate and NADPH is the reduced form of NADP⁺.

The method may include lysing the blood sample and measuring an activity of an enzyme in the lysed blood sample. A lysed blood sample is produced by treating a liquid whole blood or dried blood sample with a lysing process. Lysing the blood sample can be accomplished by any means as is known in the art. For example, lysing the blood sample is accomplished by mixing a blood sample with a lysis buffer.

In examples, the activity of the enzyme can be correlated to the hematocrit of the blood sample. In some embodiments, the activity of the enzyme is dependent on NADP⁺, for example G6PDH catalyzes NADP⁺ to NADPH.

The method may include measuring the activity of the enzyme in standard blood samples having a known hematocrit to create a standard curve and comparing the activity of the enzyme in the lysed blood sample to the standard curve to determine the hematocrit in a blood sample based on the activity of the enzyme. The activity of the enzyme in the lysed blood sample may be determined by measuring the redox products of the enzyme. For example, when the enzyme is G6PDH, the redox product may be 6-phosphogluconolactone (PGA) and NADPH

In one embodiment of the disclosure, the activity of G6PDH is measured by allowing the lysed blood sample to react for a predetermined reaction time, measuring a first absorption of the lysed blood sample at the beginning of a predetermined reaction time, measuring a second absorption of the lysed blood sample at the conclusion of the predetermined reaction time, calculating a rate of formation of NADPH based on a change in absorption between the second absorption and the first absorption, and comparing the rate of formation with a standard curve to determine the hematocrit in the blood sample. Other examples are possible.

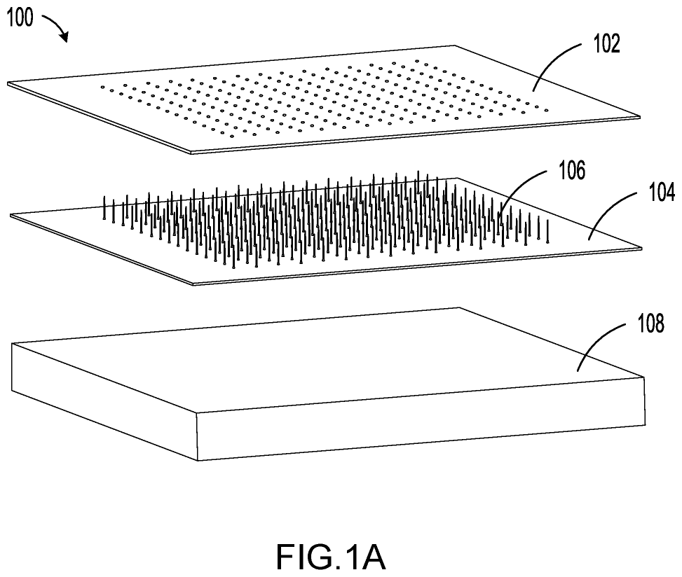

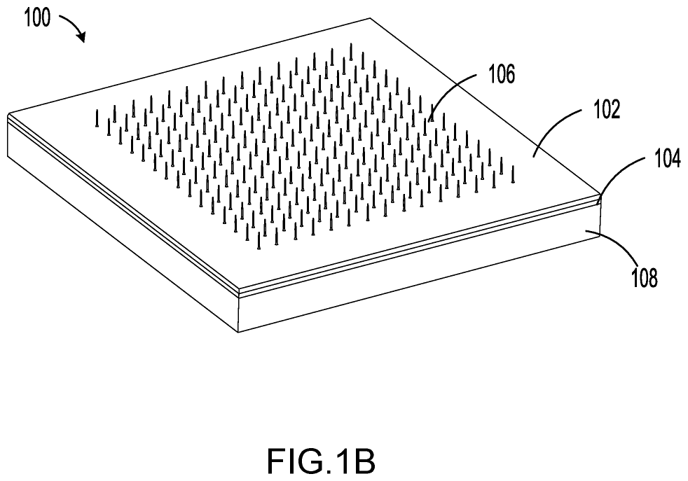

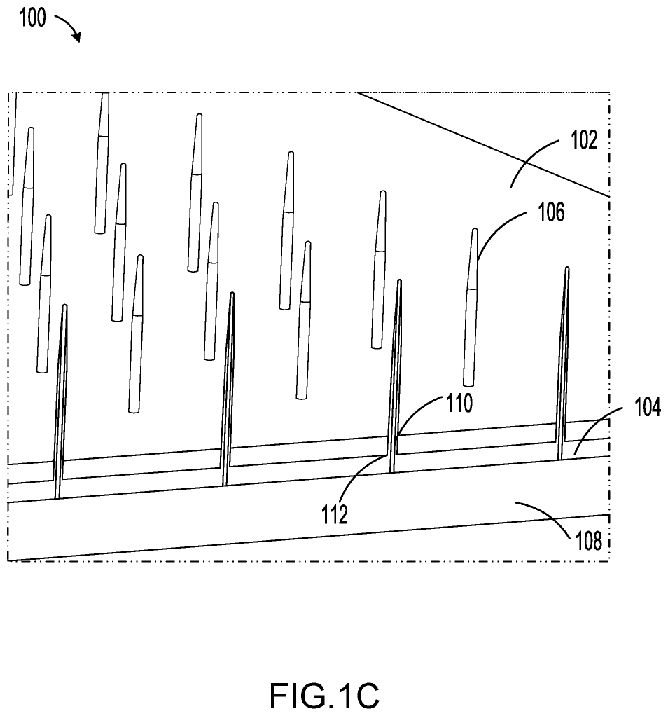

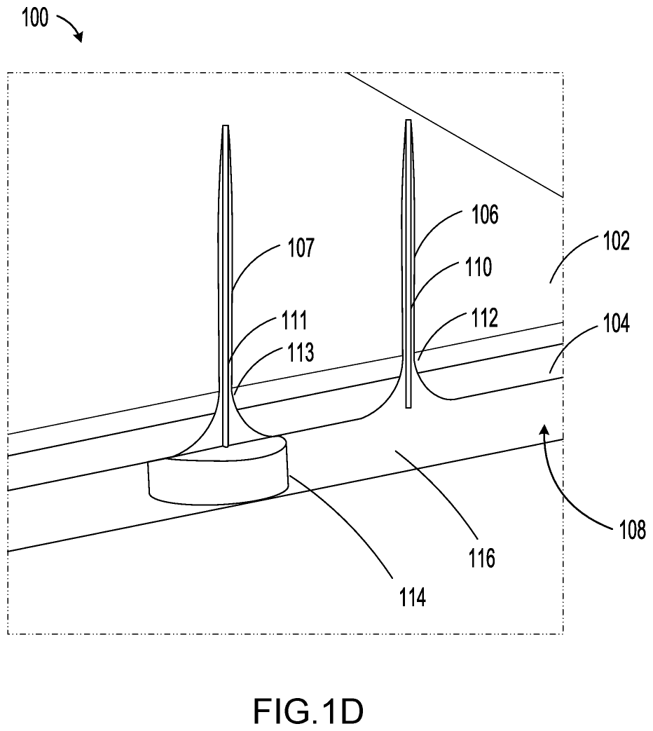

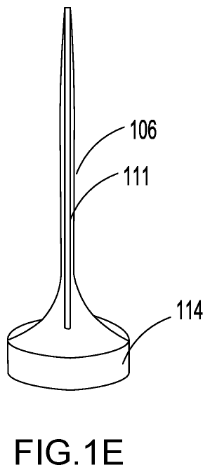

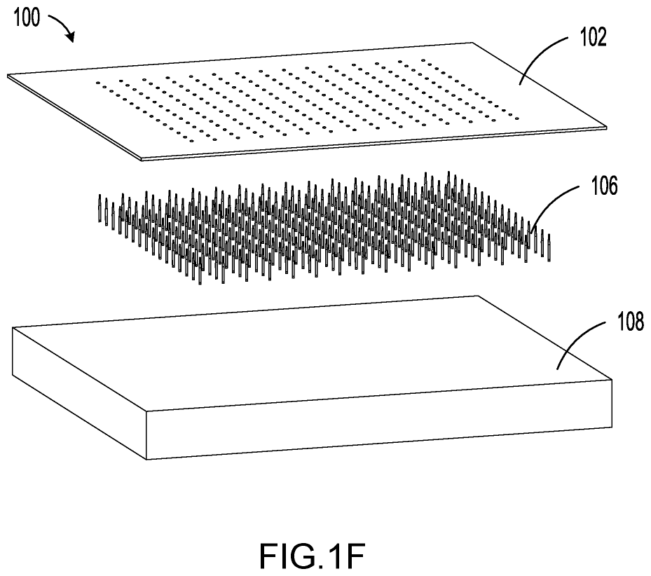

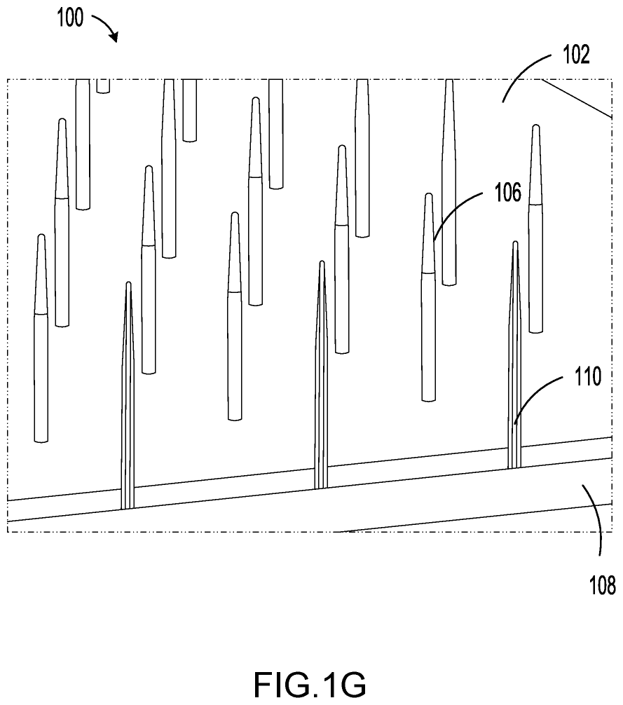

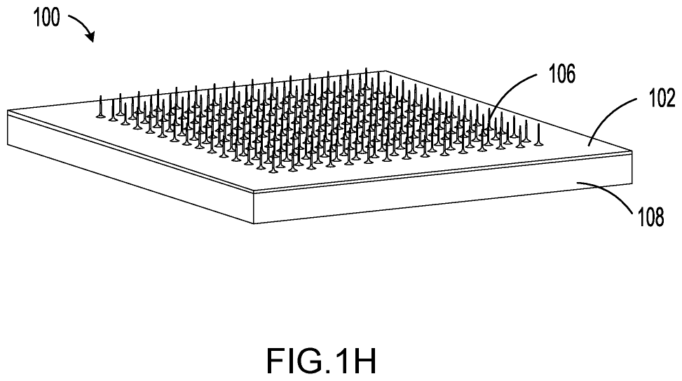

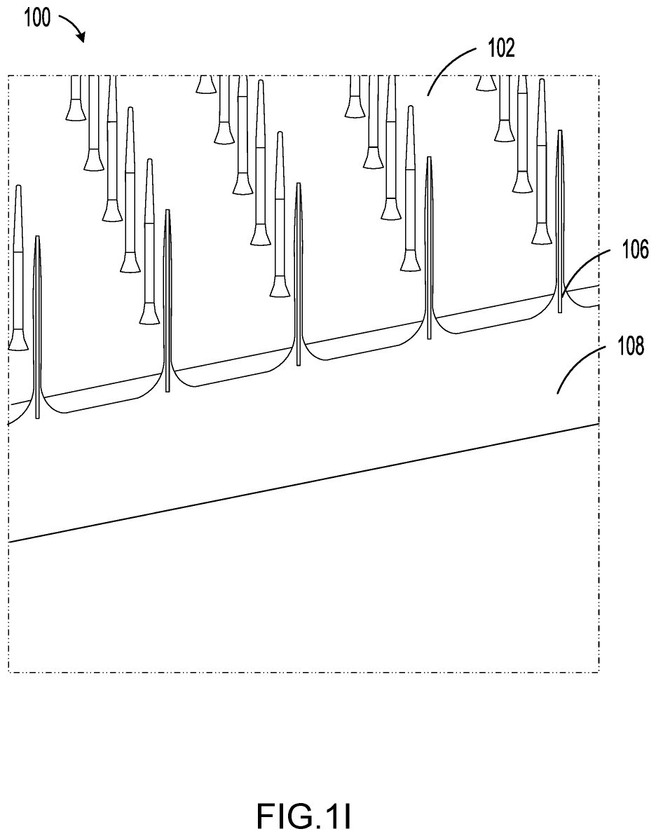

Referring now to the figures, FIGS. 1A-1I illustrate example blood collection devices 100. Specifically, FIG. 1A illustrates an exploded view of example blood collection device 100, FIG. 1B illustrates an assembled view of the blood collection device 100 of FIG. 1A, and FIG. 1C illustrates an enlarged section view of the microneedle array of FIG. 1A. FIG. 1D illustrates an enlarged section view of a microneedle array of another example blood collection device 100. FIG. 1E illustrates a microneedle in isolation that could be used in accordance with the example blood collection devices 100. FIG. 1F illustrates an exploded view of another example blood collection device 100, and FIG. 1G illustrates an assembled view of the blood collection device 100 of FIG. 1F. FIG. 1H illustrates an assembled section view of another example blood collection device 100, and FIG. 1I illustrates an enlarged section view of the microneedle array of FIG. 1H.

As shown in FIGS. 1A-1C in this example embodiment, the blood collection device 100 includes an outer layer 102, an intermediate layer 104 comprising a microneedle array 106, and blood storage layer 108. In some embodiments and in the embodiment depicted in FIGS. 1A-1C, the microneedles of the microneedle array 106 are arranged in a grid (i.e., in aligned rows and columns), however, it should be understood that the microneedles of the microneedle array 106 can be arranged in any suitable formation.

In example embodiments, the blood collection device 100 includes the outer layer 102 surrounding one or more microneedles of the microneedle array 106. In some examples, outer layer 102 may provide one or more benefits to blood collection device 100, including fortifying and/or restricting movement of the one or more microneedles with respect to the blood storage layer 108. In an example embodiment, the outer layer 102 may be made of one or more materials (e.g., a polymer material or the like) and may also be used to provide a structural characteristic to the blood collection device 100 and/or one or more components thereof. For example, the outer layer 102 may provide structural support to the blood collection device 100 and/or one or more components thereof and strengthen the blood collection device 100 or components thereof by adding rigidity to the structure. Additionally, the outer layer 102 may also be configured to wrap around one or more edges of the blood storage layer 108 to provide further structural support the blood collection device 100.

In some example embodiments, the outer layer 102 defines one or more apertures 112 extending through a thickness of the outer layer 102. Individual microneedles of the microneedle array 106 are aligned with the one or more apertures 112 of the outer layer 102. When assembled, the individual microneedles of the microneedle array 106 extend through and beyond the one or more apertures 112 of the outer layer 102, as shown in FIG. 1C. In embodiments, the one or more apertures 112 of the outer layer 102 restrict movement of the one or more microneedles of the microneedle array 106 (e.g., in a lateral and/or longitudinal direction) with respect to the outer layer 102 and with respect to one another. By restricting movement of the one or more microneedles of the microneedle array 106, the outer layer 102 may assist with restricting undesired deformation and/or fracture of the one or more microneedles of the microneedle array 106. In some examples, the outer layer 102 may cover just a portion of blood collection device 100, the portion including half of the blood collection device 100, for example. In some examples, the outer layer 102 is otherwise non-contiguous across blood collection device 100.

In some example embodiments, the outer layer 102 includes one or more additional substances positioned thereon and/or integrated therein. In some examples, outer layer 102 and/or the microneedles in microneedle array 106 includes a pharmaceutical agent positioned on and/or integrated within one or more microneedles of the microneedle array 106 and/or the outer layer 102, and that pharmaceutical agent may be transferred to the animal upon use. In some examples, this pharmaceutical agent may include an anesthetic to make the blood collection process more comfortable for the animal as compared to blood collection processes that do not include application of an anesthetic, among other possibilities. In some embodiments, the pharmaceutical agent may include an antibacterial substance or other agent to reduce the risk of infection and/or promote healing of the animal's skin. In some examples, the pharmaceutical agent may include one or more substances or other agents to impart one or more pharmacological benefits to the animal during the blood collection process (e.g., treating the animal with a steroid medication, heartworm medication, etc.).

As seen in FIG. 1C, in some embodiments, one or more microneedles of the microneedle array 106 define the hollow inner channel 110, through which blood can pass from the animal to the blood storage layer 108. In some embodiments, the hollow inner channel 110 extends through the one or more microneedles of the microneedle array 106 and through the intermediate layer 104. Blood storage layer 108, in such embodiments, includes an absorbent material configured to receive blood from the hollow inner channel 110 of one or more microneedles in microneedle array 206 and the intermediate layer 104 and retain it until the blood sample is extracted from the blood collection device later for analysis and/or testing. Other examples are possible.

Blood from the animal may also pass to the blood storage layer 108 along an outer surface of the one or more microneedles of the microneedle array 106. In some embodiments, one or more of the microneedles of the microneedle array 106 are solid and do not define the hollow inner channel 110. In these embodiments, blood from the animal can be transported to the blood storage layer 108 along the surface of the one or more microneedles of the microneedle array 106. In some embodiments, the intermediate layer 104 is permeable or semi-permeable, such that blood from the animal can pass through the intermediate layer 104. For example, in embodiments in which one or more microneedles of the microneedle array 106 are solid (i.e., do not define the channel 110), blood from the animal may pass along the outer surface of the one or more microneedles of the microneedle array 106, through the intermediate layer 104, to the blood storage layer 108.

In some examples, blood collection device 100 is arranged to extract a blood sample from one or more appendages of an animal. In example embodiments, the blood collection device 100 includes at least one microneedle in the microneedle array 106 to pierce the skin of the animal and pass the interstitial fluid layer to collect a blood sample. In some embodiments, the blood collection device 100 includes plurality of microneedles in the microneedle array 106 to pierce the skin of the animal and pass the interstitial fluid layer in order to collect a blood sample.

For example, individual microneedles of the microneedle array 106 may have a small diameter. In some embodiments, individual microneedles of the microneedle array 106 have a diameter less than about 1.0 millimeters (mm), less than about 0.5 mm, less than about 0.1 mm, less than about 50 micrometers (1 μm), less than about 25 μm, or the like. Furthermore, in some embodiments and as depicted in FIG. 1C, one or more of the microneedles of the microneedle array 106 define a hollow inner channel 110 through which blood passes from the animal to the blood storage layer 108. Due to the small size of the one or more microneedles of the microneedle array 106, the one or more microneedles may fracture and/or deform as forces are applied to the one or more microneedles of the microneedle array 106, for example as the microneedle array 106 is applied to the animal and/or as forces are inadvertently applied while handling the microneedle array 106. In some instances, fractured and/or deformed microneedles may be less effective at transporting blood to the blood storage layer 108 than microneedles that have not fractured or deformed. Accordingly, by restricting movement of the one or more microneedles of the microneedle array 106, the apertures 112 of the outer layer 102 may assist in maintaining the integrity of the one or more microneedles 106.

In example embodiments, the shape and dimensions of each microneedle of the microneedle array can vary, depending on different uses. For example, longer lengths of each microneedle may be utilized for animals with larger appendages, thicker skin, or denser fur. Other examples are possible. In embodiments, the one or more microneedles may have one or more shapes, for example and without limitation: a cylindrical shape, a conical shape, a frustoconical shape, a pyramid shape, a square prism, a pentagonal prism, a hexagonal prism, an octagonal prism, and/or an annular prism, and/or any suitable combination thereof, including, for example, a microneedle that has a cylindrical base and terminates in a conical tip. Other shapes are also possible. In one aspect, the at least one microneedle in the microneedle array 106 is in communication with the blood storage layer 108. For example, the at least one microneedle of the microneedle array 106 facilitates the transport of blood from the animal to the blood storage layer 108. In some embodiments, the microneedle array 106 is monolithic with the intermediate layer 104. In some embodiments, the microneedle array 106 is coupled to the intermediate layer 104. In embodiments, the microneedle array 106 and the intermediate layer 104 are formed of the same material or from different materials. In some examples, the microneedles in the microneedle array may comprise a silicon needle weaved into an absorbent material, like a cotton pad, of the blood collection mechanism.

In example embodiments, the shape and dimensions of different microneedles of the microneedle array can vary, depending on different uses, including within a singular microneedle of microneedle array 106. In some examples, the microneedle array 106 may be altered and/or interchanged in a variety of ways. For example, one or more microneedles in the microneedle array 106 may be added or removed from the blood collection device and/or interchanged with one or more microneedles of a different configuration, shape, etc., among other possibilities. In any event, the at least one microneedle in the microneedle array 106 may transport blood from the animal to the blood storage layer 108

In some embodiments, a user of the blood collection device 100 may apply pressure to the appendage of the animal with the blood collection device 100 until one or more designated events occur indicating a proper volume of blood has been collected to perform a blood test. In some cases, the designated event might be the passage of a predetermined amount of time. For instance, the length of time to collect a predetermined volume of blood sufficient to perform a blood test is between about 30 seconds and about 5 minutes, inclusive of the endpoints. In some embodiments, the blood storage layer 108 is structurally configured to change color when the predetermined volume of blood has been collected, for example as the result of saturation of the blood storage layer 108 and/or via interaction with the blood storage layer 108.

In example embodiments, the blood storage layer 108 is structurally configured to hold the collected blood sample until the blood sample is extracted from the blood storage layer 108 for analysis and/or testing. In some examples, the blood storage layer 108 is formed of or includes absorbent material that is designed to store blood samples, which may adhere and/or otherwise dry on the absorbent material. For example, in some embodiments, the blood storage layer 108 may include cotton, cellulose-based materials, filter paper, non-fibrous materials, or collection cards, which are typically absorbent and inert fibrous thin sheet materials, and the like and/or any suitable combination thereof. In some examples, the blood storage layer 108 define one or more chambers to store blood samples, including coagulated blood samples.

In some examples, such as depicted in FIG. 1D, a cross-section of an example blood collection device 100 is shown, according to an example embodiment. In this example embodiment, the device includes the outer layer 102, the intermediate layer 104 comprising the microneedle array comprising microneedles 106 and 107, and the blood storage layer 108 that includes a combination of an absorbent material 116 and defines one or more chambers 114 to store blood samples, among other possibilities. In example embodiments, if the blood storage layer 108 includes and/or is formed of an absorbent material and defines the at least one chamber, at least two different types of blood samples may be extracted from the blood storage layer 108 and analyzed: (i) a dried blood sample from the absorbent material portion 116 of the blood storage layer 108; and (ii) a coagulated liquid blood from the at least one chamber 114 of the blood storage layer 108. Other examples are possible.

In some examples, as illustrated in FIG. 1D, blood may be transported: (1) through aperture 112 of outer layer 102 and intermediate layer 104 via hollow inner channel 110 of a microneedle of microneedle array 106 of blood collection device 100 to the absorbent material portion 116 of blood storage layer 108 and (2) through aperture 113 of outer layer 102 and intermediate layer 104 via channel 111 of microneedle 107 of blood collection device 100 to blood storage chamber portion 114 of blood storage layer 108. In some examples, blood can also pass through or along a microneedle of microneedle array 106 to the blood storage layer 108 itself, or both. While in the embodiment depicted in FIG. 1D, the blood storage chamber 114 is shaped as a curvette, it should be understood that the blood storage chamber 114 may have any suitable shape for storing liquid blood. Further, while a single storage chamber 114 is depicted in FIG. 1D, it should be understood that the blood collection device 100 can include any suitable number of storage chambers 114 associated with one or more of the microneedles of the microneedle array 106.

FIG. 1E depicts a singular microneedle 107 (e.g., as illustrated in FIG. 1D of blood collection device 100) with channel 111, designed to drain into blood storage chamber 114. In some examples, blood storage chamber 114 may be designed to store a blood sample, including coagulated blood samples, until the blood sample need be accessed for analysis and/or testing at a later time. The singular microneedle of blood collection device 100 as depicted in FIG. 1E may be configured to be included in any embodiment in any embodiment of blood collection device 100.

FIGS. 1F-1G illustrate another embodiment of the blood collection device 100. Specifically, FIG. 1F illustrates an exploded view of example blood collection device 100, FIG. 1G illustrates an assembled view of example blood collection device 100 of FIG. 1F.

As is shown in FIGS. 1F-1G and similar to the embodiment described above and depicted in FIGS. 1A-1C, in this example embodiment, the blood collection device 100 includes an outer layer 102, the microneedles in the microneedle array 106, and the blood storage layer 108. However, in the embodiment depicted in FIGS. 1F-1G, the blood collection device 100 does not include the intermediate layer 104 (e.g., as illustrated in FIG. 1A-1C).

FIGS. 1H-1I illustrate another embodiment of the blood collection device 100. Specifically, FIG. 1H illustrates an assembled view of example blood collection device 100, and FIG. 1I illustrates an enlarged section view of example blood collection device 100 of FIG. 1H.

As is shown in FIGS. 1H-1I and similar to the embodiment described above and depicted in FIGS. 1A-1C, in this example embodiment, the blood collection device 100 includes an outer layer 102, the microneedles in the microneedle array 106, and the blood storage layer 108. However, in the embodiment depicted in FIGS. 1H-1I, the blood collection device 100 does not include the intermediate layer 104 (e.g., as illustrated in FIG. 1A-1C) and the microneedles in the microneedle array 106 are assembled into the blood storage layer 108 (i.e., the microneedle array 106 is monolithic with the blood storage layer 108).

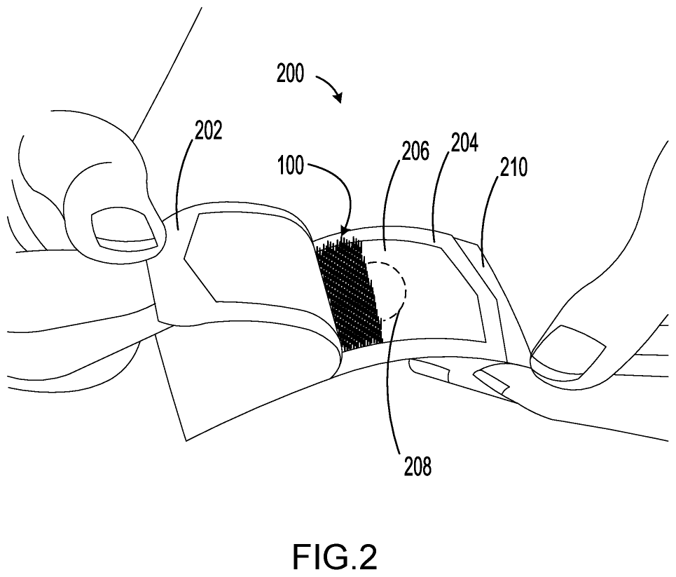

Turning to FIG. 2, in some embodiments, the blood collection device 100 further comprises a peel-to-expose package with a first tab 202 and a second tab 204. In some examples, first tab 202 and a front side of second tab 204 are configured to attach to one another when in a closed position, such as by an adhesive or other type of fastening mechanism such as a hook and loop fastener or the like. When first tab 202 is peeled away from the front side of second tab 204, the microneedle array in blood collection device 100 is exposed. In the embodiment depicted in FIG. 2, the microneedle array may be coupled to blood storage layer according to any embodiments depicted in FIGS. 1A-1I, which may in turn disposed within second tab 204. First tab 202 and second tab 204 may also be configured to re-seal to each other after the blood sample is collected and stored in blood storage layer of blood collection device 100. In some embodiment, first tab 202 may be configured to reseal to removable layer 210. In some embodiments, first tab 202 may be configured to re-seal to both second tab 204 and removable layer 210.

In example embodiments, as described above, the blood collection device may utilize an outer layer surrounding one or more microneedles of the microneedle array to provide further structural support to blood collection device 100 and/or components thereof.

For example, a back side of second tab 204 containing the blood storage layer of blood collection device 100 may be exposed by peeling back a removable layer 210. In embodiments, the second tab 204 is positioned between the removable layer 210 and the first tab 202. The removable layer 210, when closed, may protect a back side of the blood storage layer of blood collection device 100. When opened, e.g. at least partially separated from the first tab 202, the blood storage layer of blood collection device 100 is exposed from the back side, thus allowing the blood sample to be extracted from the blood storage layer for testing. In some examples, removable layer 210 may be made of a clear or transparent material to allow a user to see at least a portion of the back side of the blood storage layer of blood collection device 100 without removing the removable layer 210. In these embodiments, a user can determine that a sufficient volume of blood has been collected in the blood storage layer of blood collection device 100 based on either a change in color of the blood storage layer (e.g., as the blood storage layer is saturated with blood) or another indication by the blood storage layer.

In some embodiments, the blood collection device of FIG. 2 optionally may include a pressure indicator in the form of a compressible button 208 configured to provide haptic feedback to a user of the blood collection device. When a predetermined pressure is applied to the compressible button 208 by the user, the compressible button 208 may make an audible clicking sound and/or provide haptic feedback to alert the user that the correct pressure is being applied. The compressible button 208, in some examples, may be made of a material and in a shape so that when appropriate pressure is applied and the button is compressed, the sound is made and/or the blood sample may be visually inspected via removable layer 210, particularly if one or more portion of removable layer 210 comprise a transparent material. Further, in some examples, the compressible button 208 may be positioned in second tab 204 behind the microneedle array of blood collection device 100 so that, when the user pushes against it when drawing blood from the animal, the haptic feedback alerts the user that the device is being applied with the appropriate pressure.

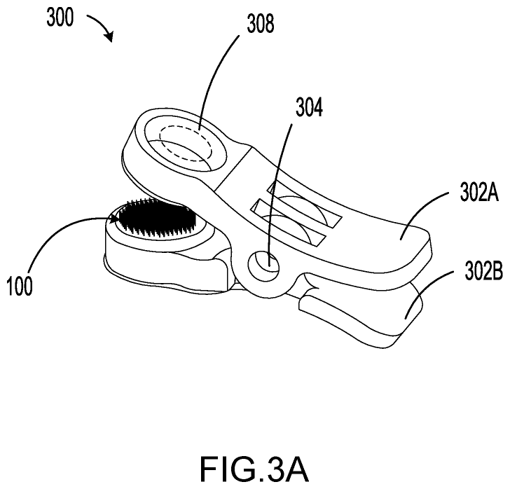

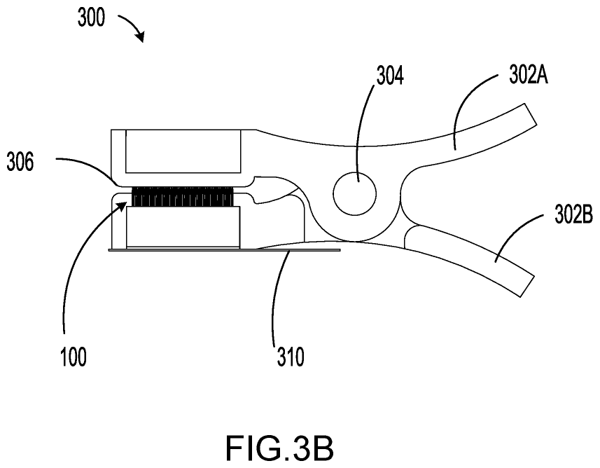

FIGS. 3A-3B depict example blood collection device 100, which may include any combination of elements shown in FIGS. 1A-1I as well as a clamping mechanism 300. As shown in the example embodiment in FIGS. 3A-3B, the clamping mechanism 300 includes a top clamp 302A and a bottom clamp 302B that oppose one another and are movable in relation to each other. In some examples, the clamping mechanism 300 also may contain a biasing member 304 coupled to the top clamp 302A and the bottom clamp 302B. The biasing member 304 can include a spring, such as a compression spring, a torsion spring, an extension spring, or the like.

In embodiments, the microneedle array of blood collection device 100 is positioned on one of the top clamp 302A or the bottom clamp 302B, and the other of the top clamp 302A or the bottom clamp 302B includes a clamping surface 306. For example, in the embodiment depicted in FIGS. 3A and 3B, the bottom clamp 302B includes the microneedle array of blood collection device 100, and the top clamp 302A includes the clamping surface 306.

In some examples, the biasing member 304 biases the microneedles of the microneedle array of blood collection device 100 into a closed position (FIG. 3B) in which the microneedle array is closer to the clamping surface 306 than in an open position (FIG. 3A).

As shown in FIG. 3B, in some embodiments, a back side of the blood storage layer of blood collection device 100 is covered by removable layer 310 that covers at least a portion of the blood storage layer. As described above, the removable layer 310 can be removed in order to access the blood sample stored in blood storage layer of blood collection device 100.

In addition, the blood collection device of FIGS. 3A-3B optionally may include a compressible button 308 to provide feedback to a user of the blood collection device that an appropriate predetermined pressure has been applied by the blood collection device 100.

In examples, when in operation, blood collection device 100 may be arranged to extract a blood sample from an appendage of an animal. In order to operate the device, the top clamp 302A and bottom clamp 302B are biased towards one another, such as by the biasing member 304. As the top clamp 302A and the bottom clamp 302B are biased toward one another, microneedles in the microneedle array of blood collection device 100 and the clamping surface 306 are biased toward one another. An appendage of the animal may then be placed between the clamping surface 306 and the microneedle array of blood collection device 100, and the biasing member 304 holds the microneedle array against the appendage of the animal.

When the blood sample collection is complete, the top clamp 302A and bottom clamp 302B may again be pushed away from one another so that the microneedles in the microneedle array of blood collection device 100 and clamping surface 306 are again oriented further away from each other in an open position and the animal appendage can be removed.

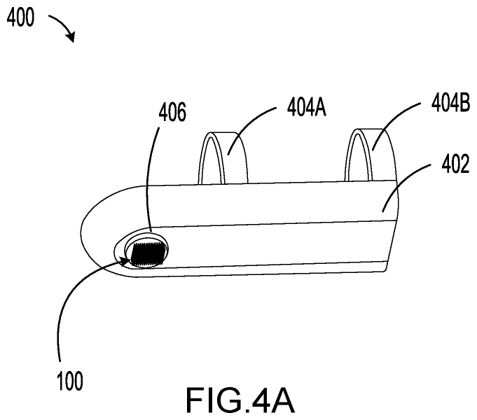

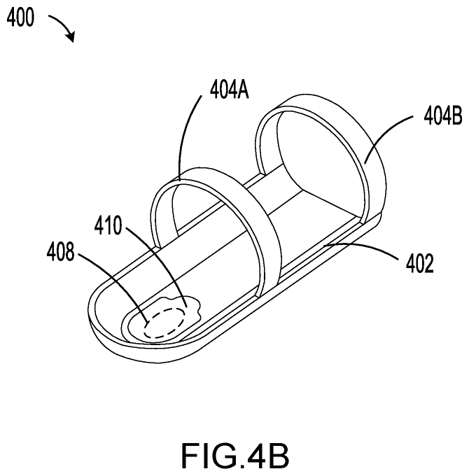

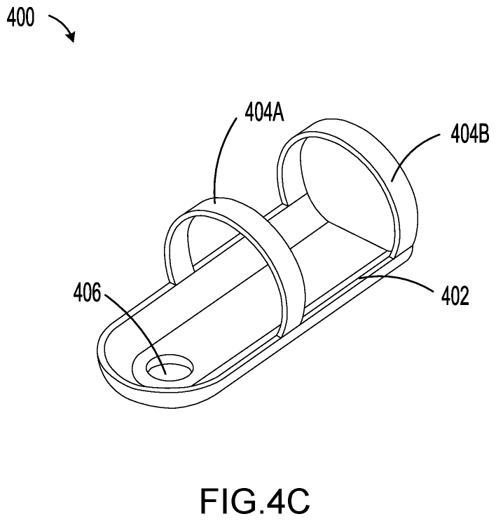

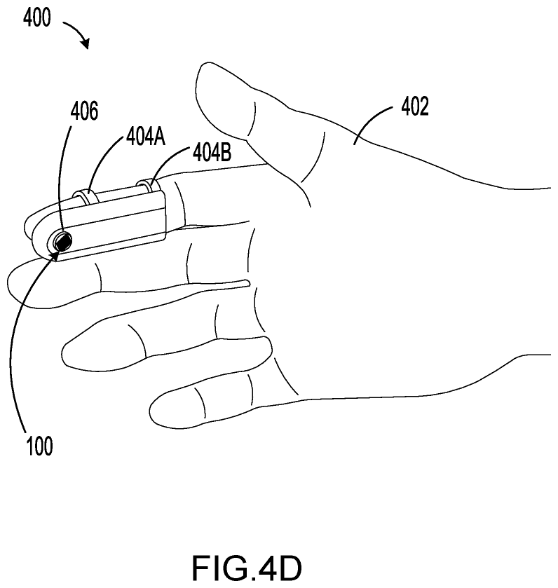

FIGS. 4A-4C depict example blood collection device 100, which may include any combination of elements shown in FIGS. 1A-1I, as well as a wearable sleeve 400. Some embodiments of the wearable sleeve 400 includes a structure suited to be worn on a user's finger (as shown in FIGS. 4A and 4B) or over a user's hand (as shown in FIG. 4C). For example, the wearable sleeve 400 includes body 402 and straps 402A and 402B, where the user's finger is configured to be guided through straps 402A and 402B with body 402 resting on a palm-side of user's finger. Wearable sleeve 400 may be suited to be worn on any finger of a user, on any particular finger of a user, on a plurality of fingers, or any combination thereof. Additionally, more straps or less straps are also possible, as well as other wearable mechanisms besides straps, such as rings or wrappers.

In embodiments, the microneedle array and blood storage layer of blood collection device 100 are positioned on an exterior surface of the wearable sleeve 400. In some embodiments in which the wearable sleeve 400 is suited to be worn on a user's finger, the microneedle array of blood collection device 100 may be configured to be positioned over the pad on a palm-side of a user's finger, facing outward.

In some embodiments, blood collection device 100 further may include a removable layer 410 on an interior surface of body 402 of the wearable sleeve 400 such that it covers an aperture 406 defined by the body 402 and/or the blood storage layer of blood collection device 100. Removable layer 410 may then expose blood storage layer of blood collection device 100 once removed at a later time so that the blood sample may be accessed for analysis and/or testing.

In addition, the blood collection device of FIGS. 4A-4C optionally may include a compressible button 408 configured to provide haptic feedback to a user of the blood collection device.

### Example Methods and Aspects

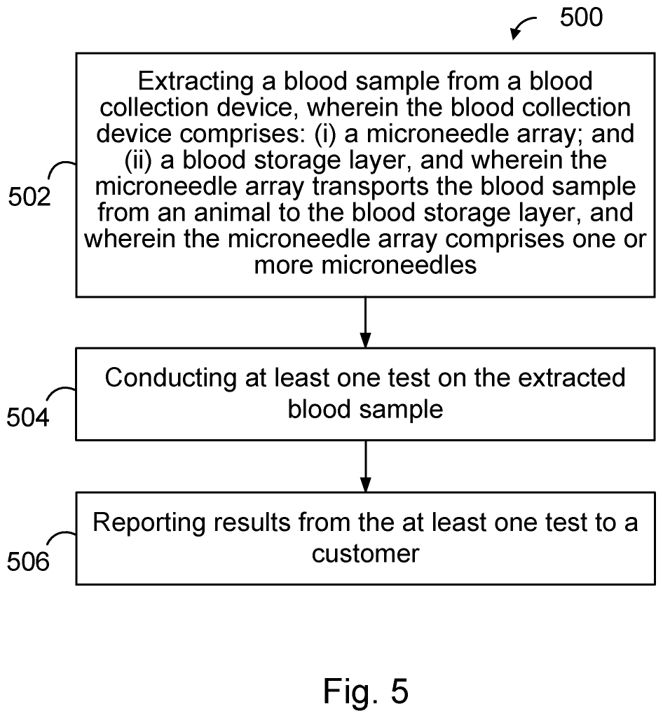

FIG. 5 shows a flowchart of an example of a method 500 for analyzing a blood sample from an animal, according to an example implementation. The method 500 shown in FIG. 5 presents an example of a method that could be used with the blood collection devices shown in FIG. 1A-4D, for example. Further, devices or systems may be used or configured to perform logical functions presented in FIG. 5. In other examples, components of the devices and/or systems may be arranged to be adapted to, capable of, or suited for performing the functions, such as when operated in a specific manner. Method 500 may include one or more operations, functions, or actions as illustrated by one or more of blocks 502-506. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 502, method 500 involves extracting a blood sample from a blood collection device 100, wherein the blood collection device 100 comprises: (i) a microneedle array 106; and (ii) a blood storage layer 108, and wherein the microneedle array 106 transports the blood sample from an animal to the blood storage layer 108, and wherein the microneedle array 106 comprises one or more microneedles. In example embodiments, the microneedle array 106 comprises a plurality of microneedles.

In example embodiments, extracting the blood sample comprises extracting the blood sample from a chamber 114 defined by the blood collection device 100. In example embodiments, extracting the blood sample comprises extracting dried blood from an absorbent material 116 of the blood storage layer. In example embodiments, extracting the blood sample comprises extracting dried blood from an absorbent material 116 of the blood storage layer 108 and further comprises extracting liquid blood from a chamber 114 defined by the blood collection device 100.

In some example embodiments, the blood collection device 100 further comprises an outer layer 102 surrounding one or more microneedles of the microneedle array 106, and wherein the outer layer 102 restricts movement of the one or more microneedles of the microneedle array 106 with respect to the blood storage layer 108. In some examples, the outer layer 102 comprises a polymer. In some examples, outer layer 102 provides a pharmaceutical agent to the animal.

In some example embodiments, the blood collection device 100 further comprises a pressure indicator, and wherein the pressure indicator comprises a compressible button (208, 308, 408, or the like) configured to provide haptic feedback to a user of the blood collection device 100 when a predetermined pressure is applied to the compressible button (208, 308, 408, or the like) by the user.

In some example embodiments, the blood collection device further comprises a peel-to-expose package 200 surrounding, at least, the microneedle array and the blood storage layer of the blood collection device 100. In example embodiments, the peel-to-expose package 200 comprises a first tab 202 that selectively covers the microneedle array of the blood collection device 100 and a second tab 204 that selectively covers the blood storage layer of the blood collection device 100. In some example embodiments, the peel-to-expose package 200 is re-sealable after the peel-to-expose package 200 is peeled away.

In some example embodiments, the blood collection device 100 further comprises a clamping mechanism 300, wherein the clamping mechanism 300: (i) injects the one or more microneedles into an appendage of the animal via a needle surface of the clamping device; and (ii) secures the microneedle array of the blood collection device 100 at a particular position in relation to the appendage of the animal via a clamping surface 306 of the clamping mechanism 300. In example embodiments, the clamping mechanism 300 includes a top clamp 302A and a bottom clamp 302B that oppose one another and are movable in relation to each other. In some example embodiments, the clamping mechanism 300 further comprises a biasing member 304 coupled to the needle surface of the blood collection device 100 and the clamping surface 306, wherein the biasing member 304 biases the microneedles of the microneedle array of blood collection device 100 into a closed position (FIG. 3B) in which the microneedle array is closer to the clamping surface 306 than in an open position (FIG. 3A).

In some example embodiments, the blood collection device 100 further comprises a wearable sleeve 400, and wherein the microneedle array and blood storage layer of blood collection device 100 are positioned on an exterior surface of the wearable sleeve 400. In example embodiments, the wearable sleeve 400 further comprises a removable layer 410 on an interior surface of the wearable sleeve 400, and wherein the removable layer 410 covers at least a portion of the blood storage layer of the blood collection device 100. In example embodiments, the blood collection device 100 further comprises a pressure indicator, and wherein the pressure indicator comprises a compressible button 408 configured to provide haptic feedback to a user of the blood collection device when a predetermined pressure is applied to the compressible button 408 by the user, and wherein the pressure indicator is positioned between the blood storage portion of the blood collection device 100 and the removable layer 410.

At block 504, method 500 involves conducting at least one test on the extracted blood sample. In example embodiments, conducting the at least one test on the extracted blood sample comprises: (i) lysing the extracted blood sample to provide a lysed blood sample; (ii) measuring an activity of G6PDH in the lysed blood sample; and (iii) correlating the activity of G6PDH in the lysed blood sample to a hematocrit of the blood sample. In some example embodiments, measuring the activity of the G6PDH comprises: (i) allowing the lysed blood sample to react for a predetermined reaction time; (ii) measuring a first absorption of the lysed blood sample at a beginning of a predetermined reaction time; (iii) measuring a second absorption of the lysed blood sample at a conclusion of the predetermined reaction time; (iv) calculating a rate of formation of NADPH based on a change in absorption between the second absorption and the first absorption; and (v) correlating the rate of formation with a standard curve to determine the hematocrit of the blood sample.

At block 506, method 500 involves reporting results from the at least one test to a customer.

In one aspect, a blood collection device for collecting a blood sample from an animal is disclosed. In example embodiments, the blood collection device includes a microneedle array, wherein the microneedle array comprises one or more microneedles. In example embodiments, the blood collection device includes a blood storage layer, wherein the microneedle array transports the blood sample from the animal to the blood storage layer. In example embodiments, the blood collection device includes an outer layer surrounding one or more microneedles of the microneedle array, and wherein the outer layer restricts movement of the one or more microneedles of the microneedle array with respect to the blood storage layer.

In one aspect, a blood collection device for collecting a blood sample from an animal is disclosed. In example embodiments, the blood collection device includes a microneedle array, wherein the microneedle array comprises one or more microneedles. In example embodiments, the blood collection device includes a blood storage layer, wherein the microneedle array transports the blood sample from the animal to the blood storage layer. In example embodiments, a wearable sleeve, wherein the sleeve injects the microneedle array into an appendage of the animal via an exterior surface of the wearable sleeve.

Various aspects of the present disclosure are presented in the following numbered clauses:

In a first aspect A1, the present disclosure provides a method of analyzing a blood sample from an animal, the method comprising: extracting a blood sample from a blood collection device, wherein the blood collection device comprises: (i) a microneedle array; and (ii) a blood storage layer, and wherein the microneedle array transports the blood sample from the animal to the blood storage layer, and wherein the microneedle array comprises one or more microneedles; conducting at least one test on the extracted blood sample; and reporting results from the at least one test to a customer.

In a second aspect A2, the present disclosure provides the method of aspect A1, wherein the microneedle array comprises a plurality of microneedles.

In a third aspect A3, the present disclosure provides the method of either of aspects A1 or A2, wherein extracting the blood sample comprises extracting the blood sample from a chamber defined by the blood collection device.

In a fourth aspect A4, the present disclosure provides the method of any of aspects A1-A3, wherein extracting the blood sample comprises extracting dried blood from an absorbent material of the blood storage layer.

In a fifth aspect A5, the present disclosure provides the method of aspect A1 wherein extracting the blood sample comprises extracting dried blood from an absorbent material of the blood storage layer and further comprises extracting liquid blood from a chamber defined by the blood collection device.

In a sixth aspect A6, the present disclosure provides the method of claim any of aspects A1-A5, wherein the blood collection device further comprises an outer layer surrounding one or more microneedles of the microneedle array, and wherein the outer layer restricts movement of the one or more microneedles of the microneedle array with respect to the blood storage layer.

In a seventh aspect A7, the present disclosure provides the method of aspect A6, wherein the outer layer comprises a polymer.

In an eighth aspect A8, the present disclosure provides the method of either of aspects A6 or A7, wherein the outer layer provides a pharmaceutical agent to the animal.

In a ninth aspect A9, the present disclosure provides the method of any of aspects A1-A8, wherein the blood collection device further comprises a pressure indicator, and wherein the pressure indicator comprises a compressible button configured to provide haptic feedback to a user of the blood collection device when a predetermined pressure is applied to the compressible button by the user.

In a tenth aspect A10, the present disclosure provides the method of any of aspects A1-A9, wherein the blood collection device further comprises a peel-to-expose package surrounding, at least, the microneedle array and the blood storage layer of the blood collection device.

In an eleventh aspect A11, the present disclosure provides the method of aspect A10, wherein the peel-to-expose package comprises a first tab that selectively covers the microneedle array and a second tab that selectively covers the blood storage layer.

In a twelfth aspect A12, the present disclosure provides the method of aspect A11, wherein the second tab comprises a transparent material.

In a thirteenth aspect A13, the present disclosure provides the method of either of aspects A11 or A12 wherein at least one of the first tab and the second tab of the peel-to-expose package is re-sealable after the peel-to-expose package is peeled away.

In a fourteenth aspect A14, the present disclosure provides the method of any of aspects A1-A13, wherein the blood collection device further comprises a clamping device, wherein the clamping device: (i) injects the one or more microneedles into an appendage of the animal via a needle surface of the clamping device; and (ii) secures the microneedle array at a particular position in relation to the appendage of the animal via a clamping surface of the clamping device.

In a fifteenth aspect A15, the present disclosure provides the method of aspect A14, wherein the needle surface and the clamping surface are opposing and positionable in relation to each other.

In a sixteenth aspect A16, the present disclosure provides the method of either of aspects A14 or A15, wherein the clamping device further comprises a biasing member coupled to the needle surface and the clamping surface, wherein the biasing member biases the needle surface and the clamping surface into a closed position, and wherein the closed position places the needle surface and the clamping surface in closer proximity than an open position of needle surface and the clamping surface.

In a seventeenth aspect A17, the present disclosure provides the method of any of aspects A1-A13, wherein the blood collection device further comprises a wearable sleeve, and wherein the microneedle array is positioned on an exterior surface of the wearable sleeve.

In an eighteenth aspect A18, the present disclosure provides the method of aspect A17, wherein the wearable sleeve further comprises a removable layer on an interior surface of the wearable sleeve, and wherein the removable layer covers at least a portion of the blood storage layer.

In a nineteenth aspect A19, the present disclosure provides the method of aspect A18, wherein the blood collection device further comprises a pressure indicator, and wherein the pressure indicator comprises a compressible button configured to provide haptic feedback to a user of the blood collection device when a predetermined pressure is applied to the compressible button by the user, and wherein the pressure indicator is positioned between the blood storage portion and the removable layer.

In a twentieth aspect A20, the present disclosure provides the method of any of aspects A1-A19, wherein conducting the at least one test on the extracted blood sample comprises: (i) lysing the extracted blood sample to provide a lysed blood sample; (ii) measuring an activity of G6PDH in the lysed blood sample; and (iii) correlating the activity of G6PDH in the lysed blood sample to a hematocrit of the blood sample.

In a twenty-first aspect A21, the present disclosure provides the method of aspect A20, wherein measuring the activity of the G6PDH comprises: allowing the lysed blood sample to react for a predetermined reaction time; measuring a first absorption of the lysed blood sample at a beginning of a predetermined reaction time; measuring a second absorption of the lysed blood sample at a conclusion of the predetermined reaction time; calculating a rate of formation of NADPH based on a change in absorption between the second absorption and the first absorption; and correlating the rate of formation with a standard curve to determine the hematocrit of the blood sample.

In a twenty-second aspect A22, the present disclosure provides a blood collection device for collecting a blood sample from an animal, comprising: a microneedle array, wherein the microneedle array comprises one or more microneedles; a blood storage layer, wherein the microneedle array transports the blood sample from the animal to the blood storage layer; and an outer layer surrounding one or more microneedles of the microneedle array, and wherein the outer layer restricts movement of the one or more microneedles of the microneedle array with respect to the blood storage layer.

In a twenty-third aspect A23, the present disclosure provides a blood collection device for collecting a blood sample from an animal, comprising: a microneedle array, wherein the microneedle array comprises one or more microneedles; a blood storage layer, wherein the microneedle array transports the blood sample from the animal to the blood storage layer; and a wearable sleeve, wherein the sleeve injects the microneedle array into an appendage of the animal via an exterior surface of the wearable sleeve.

The singular forms of the articles “a,” “an,” and “the” include plural references unless the context clearly indicates otherwise.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

What is claimed is:

1. A method of analyzing a blood sample from an animal, the method comprising:
extracting a blood sample from a blood collection device, wherein the blood collection device comprises: (i) a microneedle array; and (ii) a blood storage layer, and wherein the microneedle array transports the blood sample from the animal to the blood storage layer, and wherein the microneedle array comprises one or more microneedles;
conducting at least one test on the extracted blood sample; and
reporting results from the at least one test to a customer.

2. The method of claim 1, wherein the microneedle array comprises a plurality of microneedles.

3. The method of claim 1, wherein extracting the blood sample comprises extracting the blood sample from a chamber defined by the blood collection device.

4. The method of claim 1, wherein extracting the blood sample comprises extracting dried blood from an absorbent material of the blood storage layer.

5. The method of claim 1, wherein extracting the blood sample comprises extracting dried blood from an absorbent material of the blood storage layer and further comprises extracting liquid blood from a chamber defined by the blood collection device.

6. The method of claim 1, wherein the blood collection device further comprises an outer layer surrounding one or more microneedles of the microneedle array, and wherein the outer layer restricts movement of the one or more microneedles of the microneedle array with respect to the blood storage layer.

7. The method of claim 6, wherein the outer layer comprises a polymer.

8. The method of claim 6, wherein the outer layer provides a pharmaceutical agent to the animal.

9. The method of claim 1, wherein the blood collection device further comprises a pressure indicator, and wherein the pressure indicator comprises a compressible button configured to provide haptic feedback to a user of the blood collection device when a predetermined pressure is applied to the compressible button by the user.

10. The method of claim 1, wherein the blood collection device further comprises a peel-to-expose package surrounding, at least, the microneedle array and the blood storage layer of the blood collection device.

11. The method of claim 10, wherein the peel-to-expose package comprises a first tab that selectively covers the microneedle array and a second tab that selectively covers the blood storage layer.

12. The method of claim 11, wherein the second tab comprises a transparent material.

13. The method of claim 11, wherein at least one of the first tab and the second tab of the peel-to-expose package is re-sealable after the peel-to-expose package is peeled away.

14. The method of claim 1, wherein the blood collection device further comprises a clamping device, wherein the clamping device: (i) injects the one or more microneedles into an appendage of the animal via a needle surface of the clamping device; and (ii) secures the microneedle array at a particular position in relation to the appendage of the animal via a clamping surface of the clamping device.

15. The method of claim 14, wherein the needle surface and the clamping surface are opposing and positionable in relation to each other.

16. The method of claim 14, wherein the clamping device further comprises a biasing member coupled to the needle surface and the clamping surface, wherein the biasing member biases the needle surface and the clamping surface into a closed position, and wherein the closed position places the needle surface and the clamping surface in closer proximity than an open position of needle surface and the clamping surface.

17. The method of claim 1, wherein the blood collection device further comprises a wearable sleeve, and wherein the microneedle array is positioned on an exterior surface of the wearable sleeve.

18. The method of claim 17, wherein the wearable sleeve further comprises a removable layer on an interior surface of the wearable sleeve, and wherein the removable layer covers at least a portion of the blood storage layer.

19. The method of claim 18, wherein the blood collection device further comprises a pressure indicator, and wherein the pressure indicator comprises a compressible button configured to provide haptic feedback to a user of the blood collection device when a predetermined pressure is applied to the compressible button by the user, and wherein the pressure indicator is positioned between the blood storage portion and the removable layer.

20. The method of claim 1, wherein conducting the at least one test on the extracted blood sample comprises: (i) lysing the extracted blood sample to provide a lysed blood sample; (ii) measuring an activity of G6PDH in the lysed blood sample; and (iii) correlating the activity of G6PDH in the lysed blood sample to a hematocrit of the blood sample.

21. The method of claim 20, wherein measuring the activity of the G6PDH comprises:
allowing the lysed blood sample to react for a predetermined reaction time;
measuring a first absorption of the lysed blood sample at a beginning of a predetermined reaction time;
measuring a second absorption of the lysed blood sample at a conclusion of the predetermined reaction time;
calculating a rate of formation of NADPH based on a change in absorption between the second absorption and the first absorption; and
correlating the rate of formation with a standard curve to determine the hematocrit of the blood sample.

22. A blood collection device for collecting a blood sample from an animal, comprising:
a microneedle array, wherein the microneedle array comprises one or more microneedles;
a blood storage layer, wherein the microneedle array transports the blood sample from the animal to the blood storage layer; and
an outer layer surrounding one or more microneedles of the microneedle array, and wherein the outer layer restricts movement of the one or more microneedles of the microneedle array with respect to the blood storage layer.

23. A blood collection device for collecting a blood sample from an animal, comprising:
a microneedle array, wherein the microneedle array comprises one or more microneedles;
a blood storage layer, wherein the microneedle array transports the blood sample from the animal to the blood storage layer; and
a wearable sleeve, wherein the wearable sleeve injects the microneedle array into an appendage of the animal via an exterior surface of the wearable sleeve.
